(19) United States
(12) Patent Application Publication
(10) Pub. No.: US 2016/0060030 A1
(43) Pub. Date: Mar. 3, 2016

(54) Garbage Disposer for Multistory Building

(71) Applicant: Andrejs Saponenko, Marupe (LV)

(72) Inventor: Andrejs Saponenko, Marupe (LV)

(21) Appl. No.: 14/822,356

(22) Filed: Aug. 10, 2015

(30) Foreign Application Priority Data
Aug. 26, 2014 (RU) 2014134731

(51) Int. Cl. B65F 5/00; A61L 9/12; A61L 2/20

(52) U.S. Cl. CPC B65F 5/00; A61L 2/202; A61L 9/12

(57) ABSTRACT

A claimed useful model relates to the structures intended for utilization of domestic garbage in multistory buildings with an installation for disinfection and ozone odor control. The technical result by application of the useful model consists in the elevation of an ozone application safety by disinfection and odor control of the garbage disposers in a multistory building because of an automatic stop of an ozone supply. The technical result is attributable to the fact that the garbage disposer for a multistory building comprising the dust-collecting shaft, the disposer hopper, the dust-collecting chamber with the dustbin, the ozone generator, the methane concentration detectors, one of which is located in the dust-collecting chamber, and another one—in the exit of the airflow out of the dust-collecting shaft, is provided, in accordance with the useful model, with at least one ozone concentration detector, which is located in communal facilities adjoining with the garbage disposer and connected with the ozone generator through the regulating relay.

### CROSS-REFERENCE TO RELATED APPLICATIONS

This application claims priority to Russian Federation patent application No. RU2014134731, filed on Aug. 26, 2014, which is incorporated by reference herein in its entirety.

### BACKGROUND

Garbage disposer shafts can become dirty and require cleaning. Ozone has been used to clean garbage disposer shafts. The ozone can instantly oxidize odorous substances in the garbage disposer shaft

### BRIEF DESCRIPTION OF THE DRAWINGS

Many aspects of the present disclosure can be better understood with reference to the following drawing. The components in the drawing are not necessarily to scale, with emphasis instead being placed upon clearly illustrating the principles of the disclosure.

FIG. 1 is a diagram illustrating an example a garbage disposer.

### DETAILED DESCRIPTION

A claimed useful model relates to the structures intended for utilization of domestic garbage in multi-story buildings with an installation for disinfection and ozone odor control.

In accordance with the Resolution No. 170 of 27 th of September 2003 it is necessary to execute a cleaning and disinfection of garbage disposer shafts at least one time monthly. As the means for disinfection and odor control it is widely used the ozone. The atomic oxygen generated by ozone disintegration does instantly oxidize various odorous substances; it does not suppress other odors but does destroy bacteria and microorganisms producing these odors.

An unquestionable advantage of the ozone technologies consists in a high efficiency of their application, a low cost of material inputs for implementation and service of installations.

At the same time the ozone is a strongest oxidizer, and in the event of high concentrations—a virulent poison. In the Russian Federation there is prescribed a maximum permissible ozone concentration in an open area—0.16 mg, and in closed facilities—0.1 mg. The norms prescribed in the Russian Federation are the hardest. Therefore it is very important to provide a necessary ozone concentration level control in communal facilities.

There is currently in use a system of a household waste collection in multifamily residential buildings comprising a system of disinfection and ozone odor control (see the international application [PCT/LT2011/000001, WO 2011102701, date of international publication 25.08.2011, A61 L11/00, E04F 17/10, B65F 7/00]). This system does include an ozone generator, a central control device intended for programming and for an ozone generator function control, as well as means for a distribution of generated ozone through a garbage collecting system. In a particular case of use it can be provided with a control console in a form of an operator room connected to the system through a wired or a wireless way and destined for a supervision over and operation of the system. Depending on the dimensions of the garbage collecting system, it means of a building height and a height of garbage collecting shaft, the system can comprise several ozone generators installed in various places of the garbage collecting system and directed synchronously or separately with a possibility of ozone generation in specified time intervals and in specified dosages depending on the dimensions of the garbage collecting system, an ambient temperature and a frequency of a dustbin emptying. As a substantial disadvantage of this solution is a lack of control over a possible rise of the ozone concentration by jams in the garbage disposer shaft what can cause the consequences mentioned above.

In the patent RU 139827 (date of publication 27.04.2014, E04F17/10) the garbage disposer for a multi-story building comprises a dust-collecting shaft, disposer hoppers, a dust-collecting chamber with a dustbin, an ozone generator, and by all that the garbage disposer is provided with an airflow meter installed in the upper part of the dust-collecting shaft in an exit in atmosphere and connected through a wired or a wireless way with a regulating relay of ozone generator and/or methane concentration detectors, one of which is installed in the dust-collecting chamber and another one—in an exit of the airflow out of the dust-collecting shaft. The advantage of this solution consists in a use of the methane concentration detectors. The disadvantage consists in that it does not allow providing an automatic stop of the ozone generation in the event of exceeding of maximum permissible ozone concentration in the communal facilities. Because of mentioned combination of essential features this solution has been taken for a prototype.

The experience has proven that it is not sufficient to execute only control over an elevated ozone level, it is necessary to provide an automatic stop of its generation in the event of exceeding of maximum permissible ozone concentration in the communal facilities. The presented useful model is exactly directed on the solution of the problem mentioned above. The technical result by application of the useful model consists in the elevation of an ozone application safety by disinfection and odor control of the garbage disposers in a multi-story building because of an automatic stop of an ozone supply.

The technical result is attributable to the fact that the garbage disposer for a multi-story building comprising the dust-collecting shaft, the disposer hopper, the dust-collecting chamber with the dustbin, the ozone generator, the methane concentration detectors, one of which is located in the dust-collecting chamber, and another one—in the exit of the airflow out of the dust-collecting shaft, is provided, in accordance with the useful model, with at least one ozone concentration detector, which is located in communal facilities adjoining with the garbage disposer and connected with the ozone generator through the regulating relay.

The essence of the useful model is explained with the help of a general drawing of the garbage disposer where:

(1)—disposer hopper;

(2)—dust-collecting shaft;

(3)—dust-collecting chamber;

(4)—dustbin;

(5)—ozone generator;

(6)—lower methane concentration detector;

(7)—upper methane concentration detector;

(8)—tube for ozone supply to the dustbin;

(9)—analyzer (detector) of the ozone concentration in the air;

(10)—communal facilities.

The garbage disposer comprises the hoppers 1 installed in each storey of the building or between the stories, the dust-collecting shaft 2, the dust-collecting chamber 3, the dustbin 4 located in a lower part of the building in a closed room designated for these purposes, the ozone generator 5 generating ozone through the influence of a surface discharge of high frequency and high voltage on the air. The ozone generator 5 is equipped with the tube 8 for the ozone supply to the dustbin 4 and with the regulating relay (not shown in the Figure). In other embodiments the regulating relay can be located in another place.

The garbage disposer is provided with the methane concentration detectors 6 and 7 intended for a methane concentration control in the garbage disposer. The lower detector 6 is installed over the dustbin 4 in the dust-collecting chamber 3 and connected directly with the ozone generator 5. The upper detector 7 is installed in the exit of the airflow out of the dust-collecting shaft 2 and connected with the ozone generator 5 through the regulating relay. Such location of the methane concentration detectors 6 and 7 is conditioned by the fact that the methane is coming upwards through the shaft 2. The lower detector 6 is fixing the initial stages of garbage decay, and the upper detector 7 allows controlling a lowering of process intensity, and therefore there is provided the control over the ozone supply and its concentration.

A key distinction in comparison with the prototype is the availability of the analyzer—detector of the ozone concentration in the air 9 in the communal facilities 10. According to the expertise results the ozone is concentrated in the lower building stories because it is heavier than the air. For a correct functioning of the ozone concentration detector 9 it is to be installed in the communal facilities on the lower building stories, in the rooms adjoining with the garbage disposer. The analyzer—detector of the ozone concentration in the air 9 does automatically switch off the ozone generator 5 in the event that its concentration exceeds the maximum permissible ozone concentration.

The functioning of the structure is the following one.

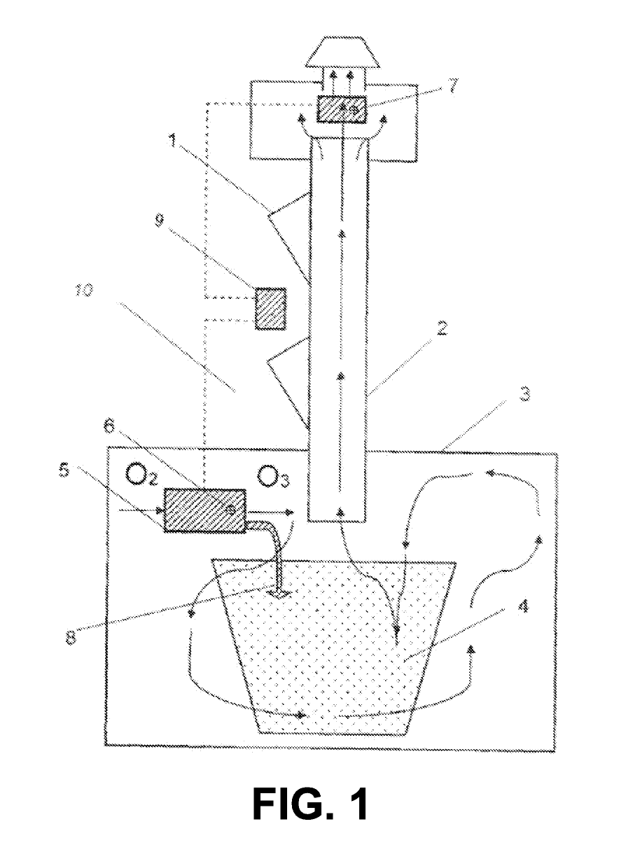

Through the hoppers 1 installed on each stories the garbage is coming to the dust-collecting shaft 2 and then to the dustbin 4 installed in the dust-collecting chamber 3. The arrows in the FIG. 1 are pointing the movement of the ozone gas from the ozone generator 5 and then upwards through the shaft 2.

The detector of the ozone concentration 9 connected with the ozone generator 5 through a bus bar is to be installed in the staircase or in another communal facilities.

The methane concentration detectors 6, 7 are controlling a methane concentration, and in the event it is close to 40 ppm they are switching on the ozone generator 5. The ozone supply is executed cyclically 1-3 times in 5- 10 minutes through the tube 8 to the dustbin 4. The cycles are to be repeated to the moment when readout of the methane concentration detector 7 shall be equal to zero. The appropriate number of cycles is depending on the number of storeys, it means on the volume of the shaft 2, as well as on climatic conditions, because an increase of the ambient temperature causes an increase of the intensity of decay process, and, consequently, an increase of the amount of generated methane.

In the event of the ozone concentration 0.09 mg (the maximum permissible ozone concentration in the Russian Federation is 0.1 mg) it shall be actuated the detector of the ozone concentration 9 which gives a signal through a wired or a wireless way to the regulating relay of the ozone generator 5 and automatically switches it off.

The ozone generation is to be stopped. By an increase of the methane concentration in the garbage disposer the methane concentration detectors 6 or 7 shall give relevant information about it and shall switch on again the ozone generator 5. The performance of the methane concentration detectors 6 and 7 and the detector of the ozone concentration 9 is synchronized with the function of the ozone generator 5 providing an ozone concentration in the dust-collecting chamber on the level of 0.3 mg and a safe maximum permissible ozone concentration in communal facilities on the level of 0.1 mg.

For an ozonization of the garbage disposer in a multifamily residential building with 9-12 stories it is reasonable to use one ozone generator 5 with a productive capacity of 300 mg per hour, and for an adequate ozonization it is necessary to apply a pulse mode, it means after 20 minutes of the generator operation to make a pause for 10-20 minutes. By the number of stories more that 12 there are to be installed two ozone generators with a regulation of the necessary capacity depending on the number of stories. The detector of the ozone concentration 9 is to be installed on the lower stories.

It is emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure.

Therefore, the following is claimed:

1. A garbage disposer for a multistory building comprising a dust-collecting shaft, a disposer hopper, a dust-collecting chamber with a dustbin, an ozone generator, methane concentration detectors, one of which is installed on the dust-collecting chamber, and another one—in an exit of airflow out of the dust-collecting shaft, characterized in that it is provided with at least one ozone concentration detector located in communal facilities adjoining with the garbage disposer and connected with the ozone generator through a regulating relay.
